(19) United States
(12) Patent Application Publication
(10) Pub. No.: US 2017/0340244 A1
(43) Pub. Date: Nov. 30, 2017

(54) AUXILIARY TESTING DEVICE

(71) Applicant: DENSO CORPORATION, Kariya-city (JP)

(72) Inventors: Shinya KUROSAWA, Kariya-city (JP); Rie OSAKI, Kariya-city (JP); Kazuo TOKUSHIMA, Kariya-city (JP)

(21) Appl. No.: 15/591,146

(22) Filed: May 10, 2017

(30) Foreign Application Priority Data
May 31, 2016 (JP) 2016-108959

(51) Int. Cl. A61B 5/097; A61B 5/093; A61B 5/03

(52) U.S. Cl. CPC A61B 5/097; A61B 5/03; A61B 5/093

(57) ABSTRACT

An auxiliary testing device assists a testing device for testing a respiratory function of a subject. The auxiliary testing device has a tubular member and a restraining member. The tubular member has a tubular shape and is connected to the testing device. The restraining member restrains a movement of a cheek of the subject.

### CROSS REFERENCE TO RELATED APPLICATION

This application is based on Japanese Patent Application No. 2016-108959 filed on May 31, 2016, the disclosure of which is incorporated herein by reference in its entirety.

### TECHNICAL FIELD

The present disclosure relates to an auxiliary testing device that assists a testing device for testing a respiratory function.

### BACKGROUND

A technique for testing a respiratory function of a subject is known. An example of the technique includes measuring a pulse wave of a subject, calculating a relative value of an amount of change in an intrathoracic pressure by using the measured value of the pulse wave, and multiplying the relative value of the amount of change by a calibration coefficient to convert the relative value into an absolute value. Patent Literature 1 (JP 2014-226422 A) discloses an example of the technique for testing a respiratory function of a subject.

The intraoral pressure is measured while the subject is breathing on a condition that the amount of change in the intrathoracic pressure is in a linear relation with an amount of change in an intraoral pressure and that a depth of respiration varies. The calibration coefficient is calculated using the measured value of the intraoral pressure. The intraoral pressure is generally measured using a mouthpiece having a pressure sensor, i.e., using the testing device.

### SUMMARY

An intraoral capacity of the subject is not fixed while the testing device measures the intraoral pressure, and thereby the intraoral capacity may vary each time the depth of respiration is changed. That is, the intraoral capacity of the subject may change each time the depth of respiration is changed and may not be able to be fixed.

When the calibration coefficient is calculated using the measured value of the intraoral pressure measured on a condition that the intraoral capacity of the subject is flexible, the calibration coefficient includes an error since the intraoral pressure is measured while the intraoral capacity varies.

Thus, the conventional technique for testing a respiratory function may not be able to measure the intraoral pressure, which is necessary for testing the respiratory function, accurately. Then, it is an objective of the present disclosure to provide an auxiliary testing device with which an intraoral pressure, which is necessary for testing a respiratory function, can be measured accurately.

An auxiliary testing device of the present disclosure assists a testing device for testing a respiratory function of a subject. The auxiliary testing device has a tubular member and a restraining member. The tubular member has a tubular shape and is connected to the testing device. The restraining member restrains a movement of a cheek of the subject.

According to the auxiliary testing device, the restraining member restrains the movement of the cheek of the subject. Accordingly, the auxiliary testing device can restrain the movement of the cheek, and thereby an intraoral capacity of the subject can be fixed, even when the subject breathes while changing a depth of the breath.

As a result, an intraoral pressure can be measured in every breath on a condition of fixing the intraoral capacity, when the subject keeps breathing while changing a depth of breath. Therefore, a measured value of the intraoral pressure measured by the testing device can have less error that occurs when the intraoral capacity is variable.

In other words, according to the auxiliary testing device of the present disclosure, the intraoral pressure and an intrathoracic pressure, which are necessary for testing a respiratory function, can be kept in a linear relation with each other and can be measured accurately.

### BRIEF DESCRIPTION OF THE DRAWINGS

The above and other objects, features and advantages of the present disclosure will become more apparent from the following detailed description made with reference to the accompanying drawings.

FIG. 1 is a diagram illustrating a schematic configuration of a respiratory function testing system.

FIG. 2 is a perspective view illustrating an auxiliary testing device on a condition that a container is shrunk, according to a first embodiment.

FIG. 3 is a perspective view illustrating the auxiliary testing device on a condition that the container expands, according to the first embodiment.

FIG. 4 is a graph showing an effect of the auxiliary testing device.

FIG. 5 is an explanatory view illustrating an auxiliary testing device according to a second embodiment.

FIG. 6 is an explanatory diagram illustrating a lock mechanism.

FIG. 7 is an explanatory view illustrating an auxiliary testing device according to a modification example of the second embodiment.

FIG. 8A is a top view illustrating an auxiliary testing device on a condition of being worn by a subject according to a third embodiment.

FIG. 8B is a side view illustrating the auxiliary testing device on the condition of being worn by the subject according to the third embodiment.

FIG. 8C is a front view illustrating the auxiliary testing device on the condition of being worn by the subject according to the third embodiment.

FIG. 9A is a top view illustrating an auxiliary testing device on a condition of being worn by a subject according to a fourth embodiment.

FIG. 9B is a side view illustrating the auxiliary testing device on the condition of being worn by the subject according to the fourth embodiment.

FIG. 9C is a front view illustrating the auxiliary testing device on the condition of being worn by the subject according to the fourth embodiment.

### DETAILED DESCRIPTION

Embodiments of the present disclosure will be described hereafter referring to drawings. In the embodiments, a part that corresponds to a matter described in a preceding embodiment may be assigned with the same reference number, and redundant explanation for the part may be omitted. When only a part of a configuration is described in an embodiment, another preceding embodiment may be applied to the other parts of the configuration. The parts may be combined even if it is not explicitly described that the parts can be combined. The embodiments may be partially combined even if it is not explicitly described that the embodiments can be combined, provided there is no harm in the combination.

### First Embodiment

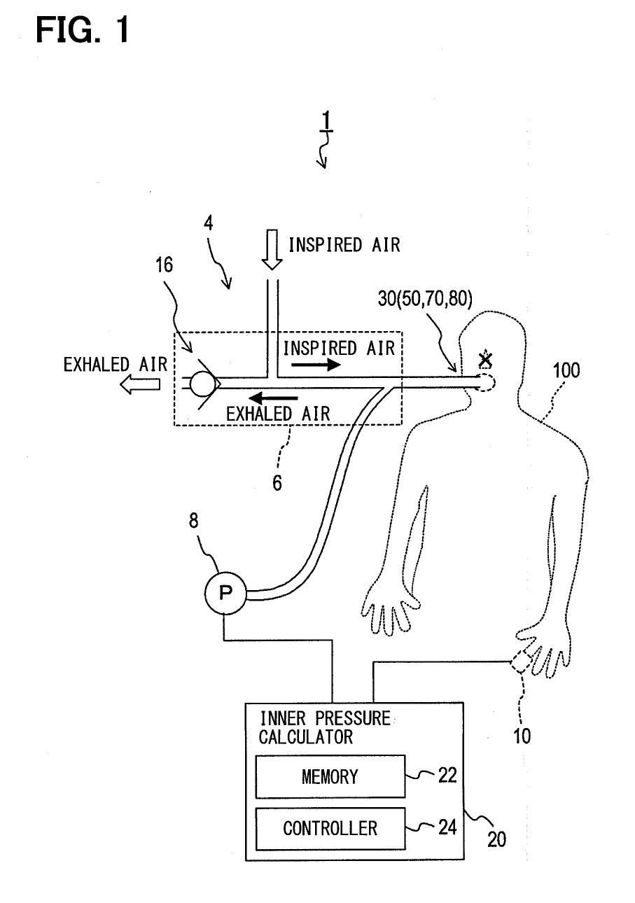

A respiratory function testing system 1 will be described referring to FIG. 1. The respiratory function testing system 1 tests a respiratory function of a subject 100.

The respiratory function testing system 1 tests a function of a respiratory apparatus of the subject 100. A test performed with the respiratory function testing system 1 includes converting an estimate value of an intrathoracic pressure, which is estimated based on a pulse wave signal corresponding to a pulse wave of the subject 100, into an absolute value of an amount of change in the intrathoracic pressure of the subject 100.

The respiratory function testing system 1 has a testing device 4 and an auxiliary testing device 30. The testing device 4 has a body 6, a pressure sensor 8, a pulse wave sensor 10, and an inner pressure calculator 20.

The body 6 has a tubular shape. An inspired air, which is drawn by the subject 100, and an exhaled air, which is discharged by the subject 100, flow in the body 6. The body 6 has an inlet from which the inspired air flows into the body 6 from an outside of the body 6. The body 6 has a first end portion in which a one-way valve 16 is disposed. The one-way valve 16 reduces an inflow amount of air flowing into the body 6 from the outside.

The pressure sensor 8 is a well-known sensor for detecting a pressure and detects an intraoral pressure of the subject 100. The pulse wave sensor 10 is a well-known sensor and detects a pulse wave of the subject 100. The pulse wave sensor 10 may be an optical sensor or a piezoelectric sensor that is worn by the subject 100.

The inner pressure calculator 20 has a memory 22 and a controller 24. The memory 22 is a rewritable non-volatile device. The memory 22 may be a hard disc (i.e., a hard drive) or a flash memory.

The controller 24 is a well-known controller that has a well-known microcomputer having ROM, RAM, and CPU. The ROM of the controller 24 stores various processing programs for performing various processing. The various processing includes calculating coefficient and calculating intrathoracic pressure.

In the calculating coefficient, a calibration coefficient is calculated. In the calculating intrathoracic pressure, an estimated intrathoracic pressure of the subject 100 is estimated based on a pulse wave signal corresponding to a pulse wave of the subject 100. The estimated intrathoracic pressure is multiplied by the calibration coefficient to be converted into an absolute value of an amount of change in the intrathoracic pressure of the subject 100.

The subject 100 breathes through the testing device 4. Air outside the body 6 flows into the body 6 through the inlet when the subject 100 breathes. The air (i.e., the inspired air) flowing into the body 6 flows into an intrathoracic space of the subject 100 through an intraoral space of the subject 100.

When the subject 100 exhales air, the air flows out of the intrathoracic space and flows into the body 6 through the intraoral space. The air (i.e., the exhaled air) flowing into the body 6 flows to an outside of the body 6 through the one-way valve 16.

The pressure sensor 8 of the testing device 4 measures a pressure of air, as the intraoral pressure, that is flowing in the body 6 while the subject 100 breathes one breath. The pulse wave sensor 10 measures a pulse wave of the subject 100 during the one breath.

The inner pressure calculator 20 uses the pulse wave signal measured by the pulse wave sensor 10 to create a first envelope that connects amplitude peaks of single pulsation, and creates a second envelope that connects peaks of the first envelope. The inner pressure calculator 20 calculates a difference between the first envelope and the second envelope as the estimated intrathoracic pressure.

The inner pressure calculator 20 multiplies the estimated intrathoracic pressure by the calibration coefficient, such that the estimated intrathoracic pressure is converted to an absolute value of an amount of change in the intrathoracic pressure. The calibration coefficient is calculated in advance of performing the test for the respiratory function. Specifically, the calibration coefficient is calculated as follows. The subject 100 breathes while changing depth of breath, and the intraoral pressure and the estimated intrathoracic pressure are measured with each breath. A slope relative to a relationship between an amount of change in the intraoral pressure with each breath and the estimated intrathoracic pressure is calculated as the calibration coefficient.

The intrathoracic pressure is a pressure in the intrathoracic space of the subject 100. The estimated intrathoracic pressure is a relative value of the intrathoracic pressure that shows a transit of a pressure as the amplitude of the pulse wave varies.

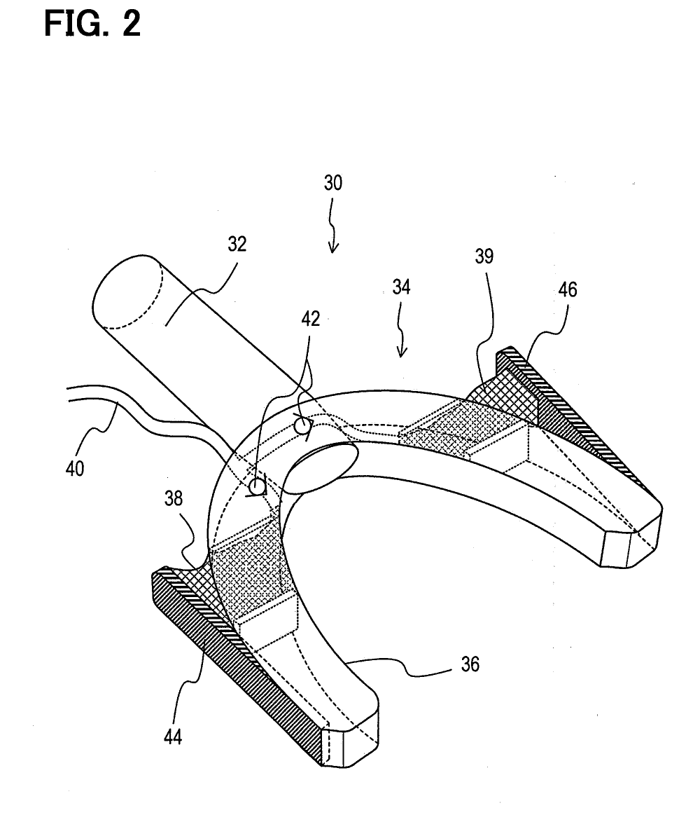

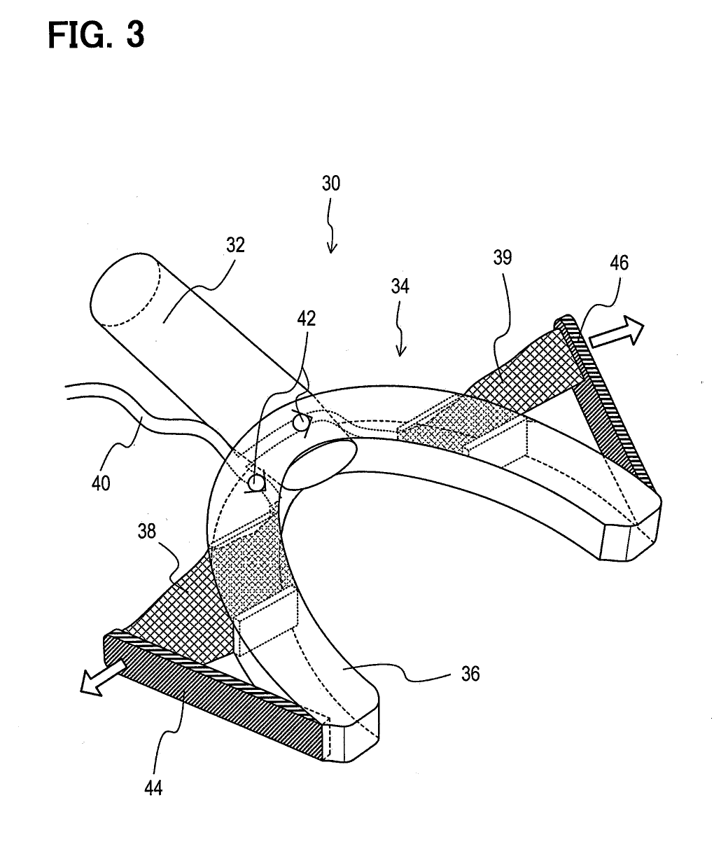

FIG. 2 and FIG. 3 illustrate the auxiliary testing device 30 that assists the testing device 4 in testing the respiratory function of the subject 100. The body 6 of the testing device 4 has the first end portion in which the one-way valve 16 is disposed and a second end portion facing the first end portion in a longitudinal direction of the body 6. According to the present embodiment, the auxiliary testing device 30 is connected to the second end portion of the body 6.

As shown in FIG. 2, the auxiliary testing device 30 has a tubular member 32 and a restraining member 34 (i.e., a cheek fixing member). The tubular member 32 has a tubular shape. The tubular member 32 has a first end portion and a second end portion facing each other in a longitudinal direction of the tubular member 32. The first end portion of the tubular member 32 is connected to the second end portion of the body 6 that is opposite to the first end portion of the body 6 in which the one-way valve 16 is disposed.

The restraining member 34 restrains a movement of cheeks of the subject 100. According to the present embodiment, the restraining member 34 supports the cheeks inside the intraoral space (i.e., inside a mouth) of the subject 100. The restraining member 34 has a base portion 36, a first container 38, a second container 39, a tube 40, a valve 42, a first pressure plate 44, and a second pressure plate 46.

The base portion 36 has an arc shape (i.e., an arched shape) and is inserted to the intraoral space of the subject 100. The base portion 36 has a through-hole that passes through the base portion 36 in a radial direction of the arc shape. The second end portion of the tubular portion 32 is inserted to the through-hole.

The first container 38 and the second container 39 are a stretch member (i.e., an elastic member). A capacity of the first container 38 and a capacity of the second container 39 increases when a fluid flows into the first container 38 and the second container 39. The first container 38 and the second container 39 may have a bag shape (i.e., a bottomed tubular shape). The tube 40 defines a passage in which the fluid flows from a pump (not shown) into the first container 38 and the second container 39. The fluid flowing into the first container 38 and the second container 39 may be air, however not limited to the air as long as being a liquid such as water that is harmless to humans. The valve 42 suppresses a backflow of the fluid from the first container 38 and the second container 39 to the pump.

The first pressure plate 44 and the second pressure plate 46 are an elongated plate member. The first pressure plate 44 is fixed to one end of the base portion 36 to extend from the one end in a normal direction of the base portion 36. The second pressure plate 46 is fixed to the other end of the base portion 36 to extend from the other end in a normal direction of the base portion 36. As shown in FIG. 3, the first pressure plate 44 and the second pressure plate 46 rotate about the one end and the other end of the base portion 36 respectively in a direction away from the base portion 36 when the capacities of the first container 38 and the second container 39 are enlarged.

Operations and effects according to the first embodiment will be described hereafter.

The second end portion of the body 6, which is opposite to the first end portion in which the one-way valve 16 is disposed, is connected with the tubular portion 32 of the auxiliary testing device 30. Then, the restraining member 34 of the auxiliary testing device 30 is inserted to the intraoral space of the subject 100.

The fluid flows into the first container 38 and the second container 39 and expands the capacities of the first container 38 and the second container 39. The first pressure plate 44 and the second pressure plate 46 are pushed by the first container 38 and the second container 39 respectively, and rotary move around the one end and the other end of the base portion 36 in the direction away from the base portion 36 respectively. As a result, the first pressure plate 44 and the second pressure plate 46 come in contact with the cheeks of the subject 100. Thus, the auxiliary testing device 30 supports the cheeks of the subject 100 inside the intraoral space.

Accordingly, the auxiliary testing device 30 can restrain a movement of the cheeks, and thereby a capacity of the intraoral space of the subject 100 can be fixed, when the subject 100 breathes while changing depth of breath.

As a result, by using the auxiliary testing device 30, the testing device 4 can measure the intraoral pressure with each breath on a condition that the capacity of the intraoral space is fixed while the subject 100 breathes with various depths of breath. That is, the auxiliary testing device 30 can reduce error in the intraoral pressure measured by the testing device 4.

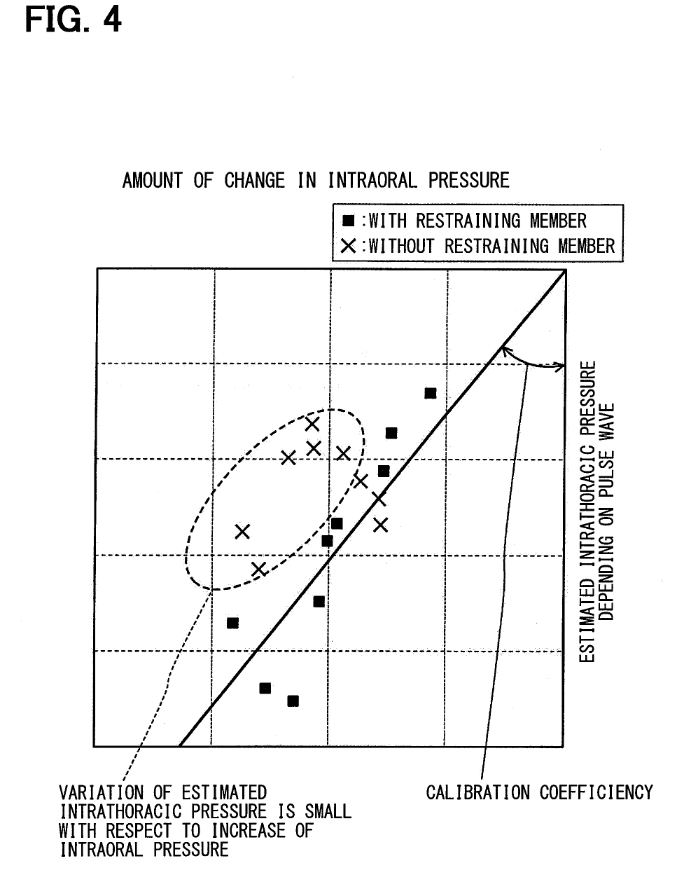

In other words, the auxiliary testing device 30 can improve accuracy for detecting the intraoral pressure that is necessary for testing the respiratory function. Thus, as shown in FIG. 4, the calibration coefficient can be calculated accurately based on the intraoral pressure that is measured using the auxiliary testing device 30.

FIG. 4 is a graph showing a relationship between the amount of change in the intraoral pressure and the estimated intrathoracic pressure estimated based on the pulse wave. FIG. 4 shows the relationship with the restraining member 34 and the relationship without the restraining member 34. The amount of change in the intraoral pressure is calculated by measuring the intraoral pressure with each breath while the subject 100 breathes with various depth of breath.

According to the graph in FIG. 4, a variation of the estimated intrathoracic pressure depending on the pulse wave is small with respect to an increase of the intraoral pressure without using the restraining member 34. In addition, linearity (i.e., linear correlation) between the amount of change in the intraoral pressure and the amount of change in the intrathoracic pressure is unstable without using the restraining member 34. On the other hand, the estimated intrathoracic pressure depending on the pulse wave varies appropriately with respect to the increase of the intraoral pressure when using the restraining member 34. This result means that the capacity of the intraoral space is fixed, and thereby the intraoral pressure, which is necessary for testing the respiratory function, can be measured accurately on a condition that the linear correlation between the intraoral pressure and the intrathoracic pressure can be kept to be stable.

Furthermore, the auxiliary testing device 30 can be inserted to the intraoral space of the subject 100 on a condition that the first container 38 and the second container 39 are shrunk. Accordingly, the auxiliary testing device 30 can be inserted to the intraoral space of the subject 100 smoothly, and the test for respiratory function can be conducted smoothly.

### Second Embodiment

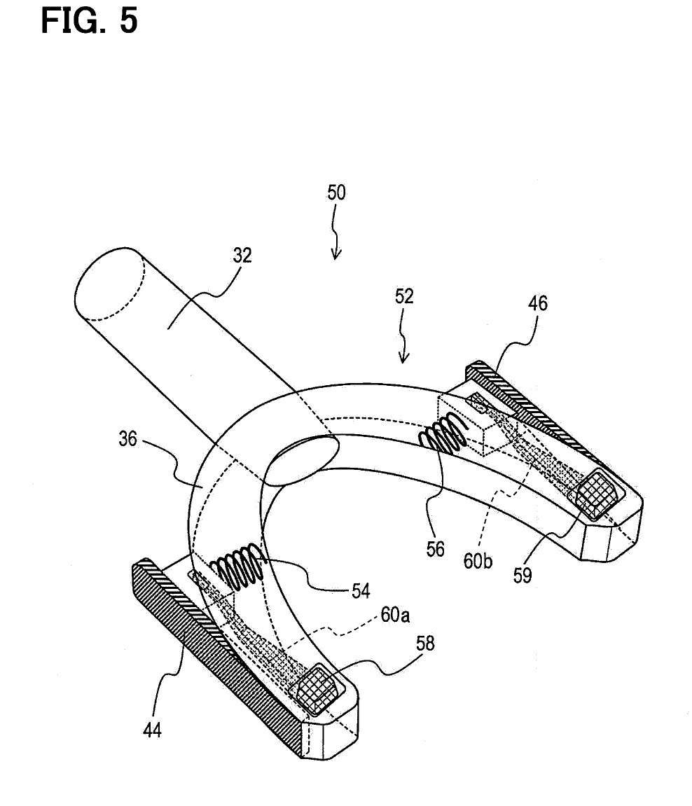

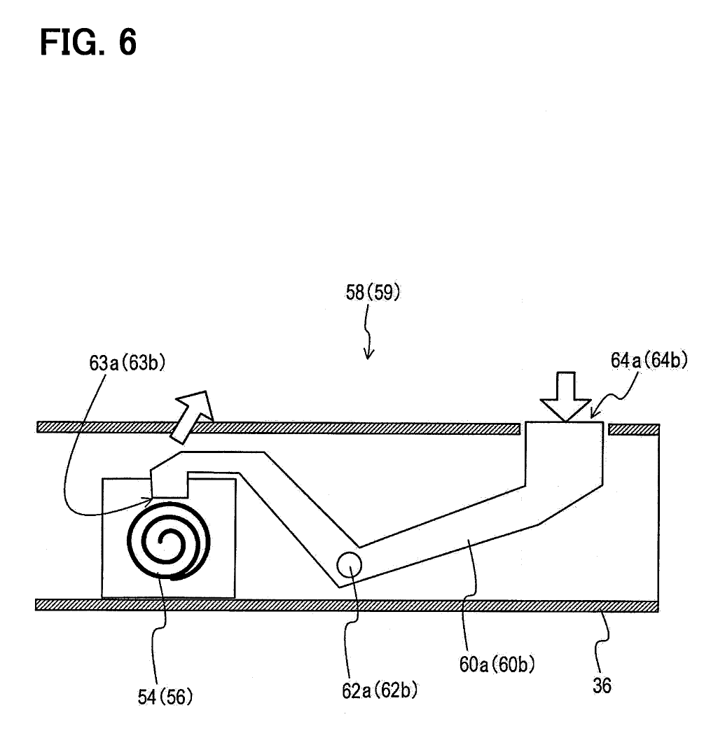

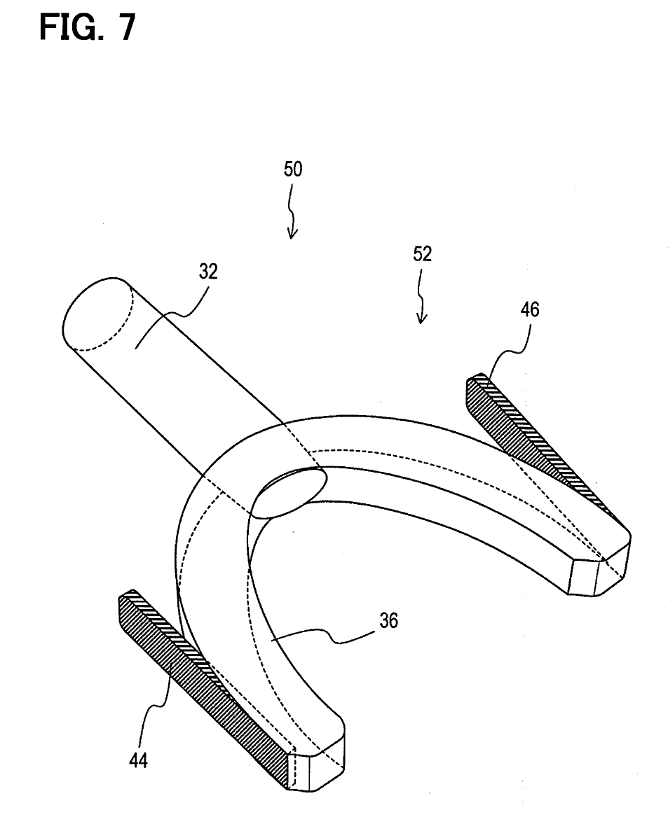

A second embodiment will be described hereafter referring to FIG. 5, FIG. 6, and FIG. 7. An auxiliary testing device 50 according to the second embodiment has a restraining member 52 of which configuration is different from that of the restraining member 34 of the first embodiment. The restraining member 52 of the second embodiment will be described mainly hereafter.

As shown in FIG. 5, the auxiliary testing device 50 has the tubular portion 32 and the restraining member 52.

The restraining member 52 restrains a movement of the cheeks of the subject 100. According to the present embodiment, the restraining member 52 supports the cheeks inside the intraoral space of the subject 100. The restraining member 52 has the base portion 36, the first pressure plate 44, the second pressure plate 46, a first bias portion 54, a second bias portion 56, a first lock mechanism 58, and a second lock mechanism 59.

The first bias portion 54 and the second bias portion 56 bias the first pressure plate 44 and the second pressure plate 46 in the radial direction of the base portion 36 away from the base portion 36 respectively. The first bias portion 54 and the second bias portion 56 are a coil spring according to the present embodiment.

As shown in FIG. 6, the first lock mechanism 58 has a first lever 60a and a first rotary shaft 62a, and the second lock mechanism 59 has a second lever 60b and a second rotary shaft 62b. The first lever 60a and the second lever 60b are an elongated member. According to the present embodiment, the first lever 60a and the second lever 60b have an S-shape.

The first lever 60a is supported by the first rotary shaft 62a located in a center portion of the first lever 60a. The first lever 60a has one end and the other end in a longitudinal direction of the first lever 60a. The one end is located between the first bias portion 54 and the first pressure plate 44, and the other end is exposed on a surface of the base portion 36, on a condition of being supported by the first rotary shaft 62a.

The second lever 60b is supported by the second rotary shaft 62b located in a center portion of the second lever 60b. The second lever 60b has one end and the other end in a longitudinal direction of the second lever 60b. The one end is located between the second bias portion 56 and the second pressure plate 46, and the other end is exposed, on a surface of the base portion 36 on a condition of being supported by the second rotary shaft 62b.

The one end of the first lever 60a and the one end of the second lever 60b will be referred to as a first acting end 63a and a second acting end 63b respectively. The other end of the first lever 60a and the other end of the second lever 60b will be referred to as a first force point end 64a and a second force point end 64b respectively.

That is, the first acting end 63a and the second acting end 63b are lifted when force is applied to the first force point end 64a and the second force point end 64b respectively. Accordingly, the first bias portion 54 and the second bias portion 56 are released, and bias the first pressure plate 44 and the second pressure plate 46 respectively outward in the radial direction of the base portion 36 away from the base portion 36.

Operations and effects according to the second embodiment will be described hereafter.

The second end portion of the body 6 of the testing device 4 is connected to the tubular portion 32 of the auxiliary testing device 50. Then, the restraining member 52 of the auxiliary testing device 50 is inserted to the intraoral space of the subject 100.

When the subject 100 presses the first force point end 64a of the first lever 60a and the second force point end 64b of the second lever 60b, the first bias portion 54 and the second bias portion 56 bias the first pressure plate 44 and the second pressure plate 46 outward respectively. For example, the subject 100 uses teeth to press the first force point end 64a and the second force point end 64b. Accordingly, the first pressure plate 44 and the second pressure plate 46 come in contact with the cheeks of the subject 100 and support the cheeks inside the intraoral space of the subject 100.

Accordingly, the auxiliary testing device 50 can restrain a movement of the cheeks, and thereby a capacity of the intraoral space of the subject 100 can be fixed, when the subject 100 breathes while changing depth of breath.

The second embodiment may be modified as follows.

The restraining member 52 has the base portion 36, the first pressure plate 44, the second pressure plate 46, the first bias portion 54, the second bias portion 56, the first lock mechanism 58, and the second lock mechanism 59. However, the first bias portion 54, the second bias portion 56, the first lock mechanism 58, and the second lock mechanism 59 may be omitted, and the restraining member 52 may have only the base portion 36, the first pressure plate 44, and the second pressure plate 46 as shown in FIG. 7. That is, the first pressure plate 44 and the second pressure plate 46 may be fixed at the one end and the other end of the base portion 36 to extend from the one end and the other end in the normal directions of the base portion 36 respectively. In this case, the first pressure plate 44 and the second pressure plate 46 function as plate springs.

The modified restraining member 52 can restrain a movement of the cheeks of the subject 100 with a simple configuration.

### Third Embodiment

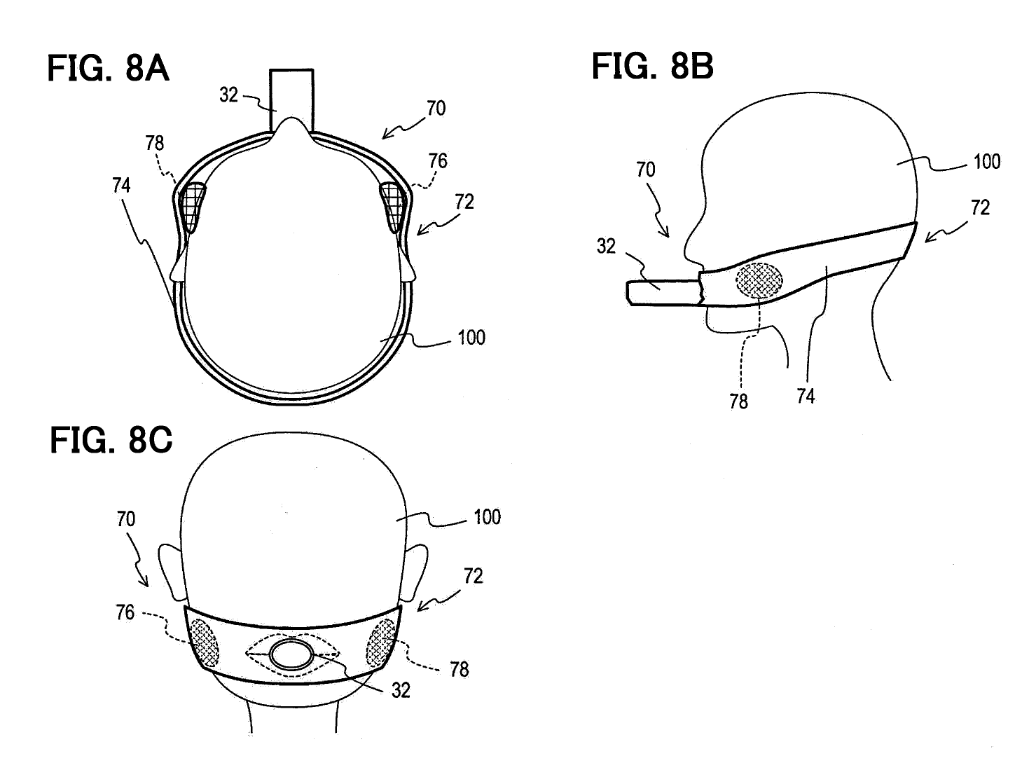

A third embodiment will be described hereafter referring to FIG. 8A, FIG. 8B, and FIG. 8C. An auxiliary testing device 70 according to the third embodiment has a restraining member 72 of which configuration is different from that of the restraining member 34 of the first embodiment and that of the restraining member 52 of the second embodiment. The restraining member 72 of the third embodiment will be described mainly hereafter.

As shown in FIG. 8A, FIG. 8B, and FIG., 8C, the auxiliary testing device 70 has the tubular portion 32 and the restraining member 72.

The restraining member 72 restrains a movement of cheeks of the subject 100. According to the present embodiment, the restraining member 72 supports the cheeks of the subject 100 outside the intraoral space of the subject 100. In other words, the restraining member 72 restrains a movement of the cheeks outside the intraoral space. The restraining member 72 has an elongated portion 74, a first container 76 and a second container 78.

The elongated portion 74 has a band shape and is attached to a head of the subject 100. The elongated portion 74 has a through-hole passing through the elongated portion 74 in a thickness direction of the elongated portion 74. The tubular portion 32 is inserted to the through-hole.

A capacity of the first container 76 and a capacity of the second container 78 increase when a fluid flows into the first container 76 and the second container 78. The first container 76 and the second container 78 may have a bag shape (i.e., a bottomed tubular shape). A fluid flows from a pump and a tube (not shown) into the first container 76 and the second container 78. The fluid flowing into the first container 76 and the second container 78 may be air, however not limited to the air as long as being a liquid such as water that is harmless to humans.

The first container 76 and the second container 78 are fixed to one surface of the elongated portion 74 on one side in the thickness direction. The one surface faces the head of the subject 100. More specifically, the first container 76 and the second container 78 are located to face the cheeks of the subject respectively.

Operations and effects according to the third embodiment will be described hereafter.

The second end portion of the body 6 of the testing device 4, which faces the first end portion in which the one-way valve 16 is disposed, is connected with the first end portion of the tubular portion 32 of the auxiliary testing device 70. Then, the second end portion of the tubular portion 32 is inserted to the intraoral space of the subject 100. The elongated portion 74 is attached to the head of the subject 100, and the fluid is supplied into the first container 76 and the second container 78. The first container 76 and the second container 78 expand with the fluid and press the cheeks of the subject 100 from outside.

Accordingly, the cheeks of the subject 100 is fixed from outside. Therefore, the auxiliary testing device 70 can restrain a movement of the cheeks, and thereby a capacity of the intraoral space of the subject 100 can be fixed, when the subject 100 breathes while changing depth of breath.

In other words, the auxiliary testing device 70 can measure the intraoral pressure, which is necessary for testing the respiratory function, accurately.

### Fourth Embodiment

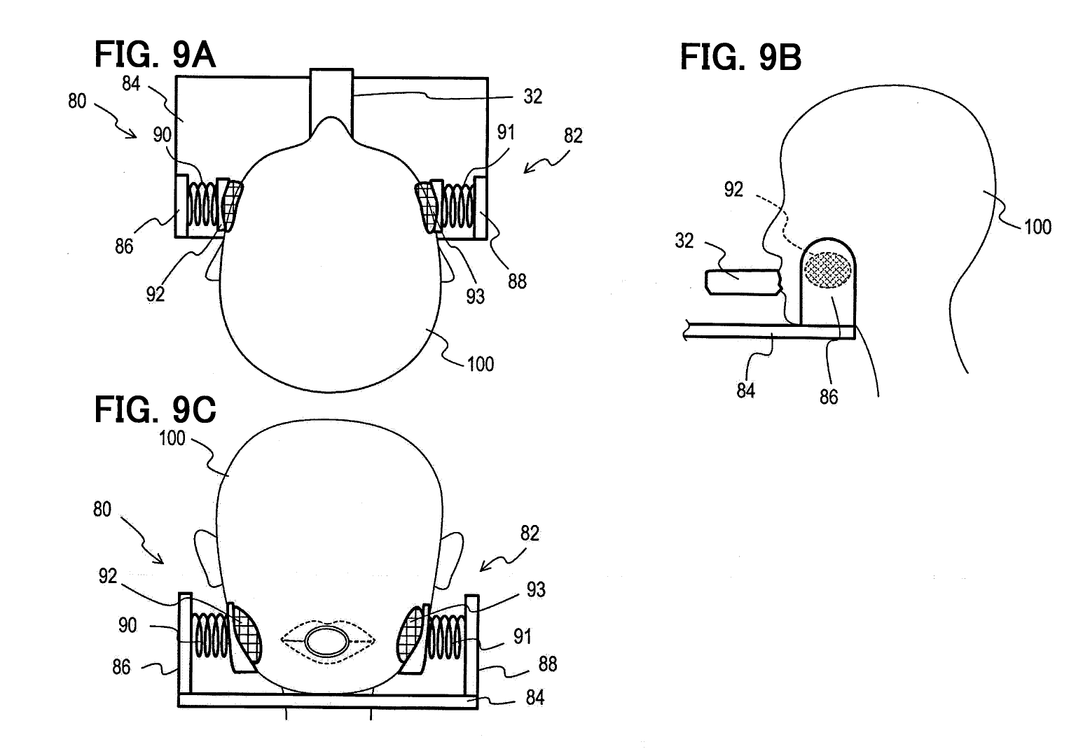

An auxiliary testing device 80 of a fourth embodiment will be described referring to FIG. 9A, FIG. 9B, and FIG. 9C. The auxiliary testing device 80 has a restraining member 82 of which configuration is different from that of the restraining member 72 of the third embodiment.

As shown in FIG. 9A, FIG. 9B, and FIG. 9C, the auxiliary testing device 80 has the tubular portion 32 and the restraining member 82.

The restraining member 82 restrains a movement of cheeks of the subject 100. The restraining member 82 supports the cheeks of the subject 100 outside of the intraoral space. The restraining member 82 has a base plate 84, a first plate 86, a second plate 88, a first bias portion 90, a second bias portion 91, a first pad 92, and a second pad 93.

The base plate 84 has a plate shape. According to the present embodiment, the base plate 84 has a rectangular plate shape. The first plate 86 and the second plate 88 have a plate shape.

The base plate 84 has a first side portion and a second side portion facing each other and parallel to each other. As shown in FIG. 9C, the first plate 86 is coupled with the first side portion, and the second plate 88 is coupled with the second side portion, such that the first plate 86 and the second plate 88 face each other, are parallel to each other, and are distanced from each other. The first plate 86 and the second plate 88 extend from the base plate 84 such that a head of the subject 100 can be placed between the first plate 86 and the second plate 88.

The first bias portion 90 and the second bias portion 91 are a member, which generates bias force, such as a coil spring. The first plate 86 has one surface that faces one surface of the second plate 88. The first bias portion 90 is fixed to the one surface of the first plate 86, and the second bias portion 91 is fixed to the one surface of the second plate 88. The first bias portion 90 generates bias force toward the second plate 88, and the second bias portion 91 generates bias force toward the first plate 86. That is, a direction in which the first bias portion 90 generates the bias force is opposite to a direction in which the second bias portion 91 generates the bias force.

The first pad 92 and the second pad 93 are a shock-absorbing material. The first pad 92 is fixed to an end portion of the first bias portion 90 on an opposite side of the first plate 86 with respect to the first bias portion 90. The second pad 93 is fixed to an end portion of the second bias portion 91 on an opposite side of the second plate 88 with respect to the second bias portion 91.

Effects provided by the fourth embodiment will be described hereafter.

The second end portion of the body 6 of the testing device 4, which faces the first end portion in which the one-way valve 16 is disposed, is connected with the first end portion of the tubular portion 32 of the auxiliary testing device 80. Then, the second end portion of the tubular portion 32 is inserted to the intraoral space of the subject 100. The head of the subject 100 is placed between the first plate 86 and the second plate 88. The first bias portion 90 and the second bias portion 91 push the first pad 92 the second pad 93 respectively against the cheeks of the subject 100.

Accordingly, the cheeks of the subject 100 are supported outside the intraoral space. Therefore, the auxiliary testing device 80 can restrain a movement of the cheeks, and thereby a capacity of the intraoral space of the subject 100 can be fixed, when the subject 100 breathes while changing depth of breath.

In other words, the auxiliary testing device 80 can measure the intraoral pressure, which is necessary for testing the respiratory function, accurately.

(Other Modification)

While the present disclosure has been described with reference to preferred embodiments thereof, it is to be understood that the disclosure is not limited to the preferred embodiments and constructions. The present disclosure is intended to cover various modification and equivalent arrangements within a scope of the present disclosure. It should be understood that structures described in the above-described embodiments are preferred structures, and the present disclosure is not limited to have the preferred structures.

The scope of the present disclosure includes all modifications that are equivalent to descriptions of the present disclosure or that are made within the scope of the present disclosure.

What is claimed is:

1. An auxiliary testing device that assists a testing device for testing a respiratory function of a subject, the auxiliary testing device comprising:
a tubular member that has a tubular shape and is connected to the testing device; and
a restraining member that restrains a movement of a cheek of the subject.

2. The auxiliary testing device according to claim 1, wherein
the restraining member restrains the movement of the cheek in a mouth of the subject.

3. The auxiliary testing device according to claim 2, wherein
the restraining member has
a base portion to be located inside the mouth and
a container that expands outward from the base portion when a fluid flows into the container.

4. The auxiliary testing device according to claim 2, wherein
the restraining member has
a base portion to be located inside the mouth and
a bias portion that generates a bias force in a direction away from the base portion.

5. The auxiliary testing device according to claim 1, wherein
the restraining member restrains the movement of the cheek outside the mouth.

6. The auxiliary testing device according to claim 5, wherein
the restraining member has
an elongated portion that has an elongated shape and
a container that is disposed on one surface of the elongated portion and expands when a fluid flows into the container.

7. The auxiliary testing device according to claim 5, wherein
the restraining member has
a first plate that has a plate shape and
a second plate that has a plate shape, the second plate being located to be distanced from the first plate and to face the first plate.

8. The auxiliary testing device according to claim 7, wherein
the first plate has a surface that faces the second plate, and the surface has a first bias portion that generates a bias force toward the second plate, and
the second plate has a surface that faces the first plate, and the surface has a second bias portion that generates a bias force toward the first plate.
